(19) United States
(12) Patent Application Publication
(10) Pub. No.: US 2010/0274115 A1
(43) Pub. Date: Oct. 28, 2010

(54) LEAD EXTENSION WITH PHYSIOLOGICAL DATA RECORDER

(75) Inventors: Jonathan C. Werder, Corcoran, MN (US); Randy M. Jensen, Hampton, MN (US); Donald L. Deyo, Roseville, MN (US)

Correspondence Address: SHUMAKER & SIEFFERT , P.A, 1625 RADIO DRIVE , SUITE 300, WOODBURY, MN, 55125, US

(73) Assignee: Medtronic, Inc.

(21) Appl. No.: 12/429,921

(22) Filed: Apr. 24, 2009

(51) Int. Cl. A61B 5/0476

(52) U.S. Cl. 600/378

(57) ABSTRACT

A lead extension is provided that includes a physiological data recorder configured to store physiological data from the patient. A first extension segment may electrically and physically couple an implantable medical lead to the physiological data recorder, and a second extension segment may electrically and physically couple an implantable medical device (IMD) or a secondary lead extension to the physiological data recorder. The physiological data recorder may include a processor that collects the physiological data derived from sensed electrical signals from the medical lead and a memory to store the physiological data. The physiological data recorder may also wirelessly transmit the physiological data to an external programmer, or be explanted for data retrieval. In some examples, the physiological data recorder may be powered by electrical signals generated by the IMD, which may be either signals intended solely for charging, or signals intended for stimulation therapy.

### TECHNICAL FIELD

The invention relates to medical devices and, more particularly, to devices that record data from a patient.

### BACKGROUND

Medical devices, such as implantable medical devices (IMDs), may be used to deliver therapy to patients to treat a variety of symptoms or conditions, such as chronic pain, tremor, Parkinson's disease, dystonia, epilepsy, depression, obsessive compulsive disorder, other psychological disorders, neuralgia, urinary or fecal incontinence, sexual dysfunction, sleep dysfunction, obesity, or gastroparesis. An example of a therapy delivered by medical devices to patients is electrical stimulation therapy. A medical device may deliver stimulation therapy via electrodes located proximate to or within targeted tissue for the electrical stimulation, such as the spinal cord, peripheral nerves or nervous tissue, the brain, other organs, or muscle tissue.

An IMD is typically implanted within the patient at a site separate or remote from the target tissue to be stimulated. In such cases, one or more implantable medical leads may be implanted within the patient. A proximal end of a lead may be coupled to the IMD, while a distal portion or end includes one or more electrodes and is placed proximate to or within the target tissue to be stimulated. Although the implantable medical leads may be directly coupled to the IMD, a lead extension is sometimes used to connect medical leads to the IMD for implantation flexibility and/or to facilitate greater distance between the target tissue and the IMD implant site.

In addition to delivering electrical stimulation, electrodes on implantable medical leads may be used to sense physiological signals of a patient. The electrodes used for sensing may be the same as or different than the electrodes used for delivery of stimulation. Some implantable medical leads include sensors in addition to electrodes, such as sensors configured to detect temperature, pressure, pH, vibration, motion, activity, posture or any other patient physiological condition.

Some IMDs are configured to process sensed signals. Such IMDs may evaluate, initiate, terminate, or modify a therapy, such as electrical stimulation therapy, based on such sensed signals. In some cases, an IMD may additionally or alternatively store sensed signals or information derived therefrom in a memory, and/or transmit such signals or information to devices external to the patient, such as an external programming device, via telemetry. The other device may further process the signal and/or present it to a user, for evaluation and/or control of therapy. Other IMDs are not configured to receive, store, or process signals sensed by electrodes or other sensors on the leads coupled to the IMD.

### SUMMARY

The disclosure describes lead extensions that include a physiological data recorder configured to store physiological data from the patient. Conventional lead extensions include conductors to conduct signals between a lead and an implantable medical device (IMD), thereby allowing the IMD to be implanted further from a target stimulation and/or sensing location than would be permitted by the length of the lead alone. The lead extensions described herein additionally include a physiological data recorder that is configured to store patient physiological data received via the one or more leads that are coupled to the IMD via the extension.

The physiological data recorder may provide such functionality with respect to sensed signals available from one or more leads in situations in which an IMD is not configured to provide such functionality. Explantation of an IMD may be undesirable. In situations in which an IMD is not configured to receive physiological signals from a lead and physiological sensing is desired, rather than explant and replace the IMD with a new IMD configured to provide such functionality, a lead extension with a physiological data recorder according to the disclosure may be implanted, e.g., as a replacement for an existing lead extension.

The lead extension may include a first extension segment configured to physically and electrically couple an implantable medical lead or another lead extension to the physiological data recorder. The lead extension may also include a second extension segment configured to physically and electrically couple the physiological data recorder to an IMD or another lead extension. The physiological data recorder may include a processor that generates physiological data that represents or is derived from physiological signals received from electrodes or a sensor on the implanted medical lead. The physiological data recorder or the extension including the recorder may additionally include one or more electrodes or other sensors, and the processor may similarly generate physiological data based on signals received from such additional electrodes or sensors. The data recorder also includes a memory to store the physiological data. The memory may be of sufficient size to store physiological data generated over several days, weeks, months, or even years.

The physiological data recorder may also wirelessly transmit the physiological data to an external programmer or other external device while implanted within the patient. Alternatively, the data recorder may transmit the physiological data to an external device when explanted, e.g., via a wired connection or other electrical contact with the external device.

In some examples, the physiological data recorder may include a battery that provides power for the data recorder. The battery may be transcutaneously recharged by an external device. In other examples, the physiological data recorder may be powered by electrical signals generated by the IMD, which may be signals intended solely for charging the recorder or signals intended for stimulation therapy. The recorder may include a capacitor or relatively small battery to store the energy received from the IMD. A data recorder with a power source that is rechargeable by an external source or the IMD may be constructed with a relatively small size, which may be advantageous for implantation in the patient.

In one example, the disclosure provides an implantable lead extension comprising a first extension segment, a second extension segment, and a physiological data recorder electrically and physically coupled to the first and second extension segments. The first extension segment is configured to electrically and physically couple the physiological data recorder to one of an implantable medical device (IMD) or another lead extension. The second extension segment is configured to electrically and physically couple the physiological data recorder to one of an implantable medical lead having at least one lead electrode or the other lead extension. The physiological data recorder comprises a memory, a communication module, and a processor. The processor is configured to receive a physiological signal of a patient via the at least one electrode of the implantable medical lead, generate physiological data based on the physiological signal, store the physiological data within the memory, and provide the stored physiological data to an external device via the communication module.

In another example, the disclosure provides a method comprising receiving a physiological signal via at least one electrode of an implanted medical lead with an implanted physiological data recorder of an implanted lead extension, wherein a first extension segment of the extension is coupled to the implanted medical lead and a second extension segment of the extension is coupled to an implanted medical device that delivers electrical stimulation therapy via the implanted lead, and generating physiological data with the physiological data recorder based on the physiological signal. The method further comprises storing the physiological data in a memory of the physiological data recorder, and providing the stored physiological data from the physiological data recorder to an external device.

In another example, the disclosure provides a system comprising an implantable medical lead having at least one electrode, an implantable medical device (IMD) configured to deliver electrical stimulation therapy to a patient via the at least one electrode of the lead, and an implantable lead extension. The implantable lead extension comprises a first extension segment, a second extension segment, and a physiological data recorder electrically and physically coupled to the first and second extension segments. The first extension segment is configured to electrically and physically couple the physiological data recorder to one of the IMD or another lead extension. The second extension segment is configured to electrically and physically couple the physiological data recorder to one of the implantable medical lead or the other lead extension. The physiological data recorder comprises a memory, a communication module, and a processor configured to receive a physiological signal of a patient via the at least one electrode of the implantable medical lead, generate physiological data based on the physiological signal, store the physiological data within the memory, and provide the stored physiological data to an external device via the communication module.

The disclosure provides a lead extension that may allow physiological data recording not available by the IMD which delivers therapy. In this manner, the lead extension may allow for physiological sensing for, as an example, therapy efficacy evaluation and objectification, without replacing an IMD or an implantable medical lead. In addition, the physiological data recorder may be powered by the IMD or an external source to reduce size and eliminate the need for larger, primary power sources. Further, the physiological data recorder may include electrodes or sensors in addition to those provided by the implanted lead to provide additional sensing capabilities, e.g. electrocardiogram sensing, electroencephalogram sensing, pressure sensing, or temperature sensing.

The details of one or more examples according to this disclosure are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example deep brain stimulation system that includes an example lead extension having a physiological data recorder.

FIG. 2 is a conceptual diagram illustrating an example spinal cord stimulation system that includes an example lead extension having a physiological data recorder.

FIG. 3 is a conceptual diagram illustrating an example lead extension having two extension segments coupled to one side of a physiological data recorder.

FIG. 4 is a conceptual diagram illustrating an example lead extension having two extension segments coupled to opposite sides of a physiological data recorder.

FIG. 5 is functional block diagram illustrating components of an embodiment of an example implantable medical device.

FIG. 6 is functional block diagram illustrating components of an example physiological data recorder powered by electrical signals from a stimulation generator of the implantable medical device.

FIG. 7 is functional block diagram illustrating components of an embodiment of an external programmer.

FIG. 8 is a flow diagram illustrating an example technique for connecting a lead extension having a physiological data recorder to a lead and implantable medical device.

FIG. 9 is a flow diagram illustrating an example technique for a physiological data recorder to enter a sensing mode in response to detecting stimulation therapy signals from the implantable medical device.

FIG. 10 is a flow diagram illustrating an example technique for recording physiological data with a physiological data recorder and wirelessly transferring the data from the recorder to an external device.

### DETAILED DESCRIPTION

Conventional lead extensions are commonly used to connect an implantable medical lead to an implantable medical device (IMD) implanted at a location different than the implantable medical lead. The lead extension may allow the clinician to place the IMD in a location of the patient able to support the implantation of IMD (e.g., under chest tissue, in the lower abdomen, or upper buttocks). In addition to connecting the medical lead to the IMD, a lead extension as described herein provides a physiological data recorder that stores physiological data of the patient.

A lead extension with a physiological data recorder may provide supplementary functionality to a system including the IMD. The IMD may or may not be capable of receiving, processing or storing physiological signals sensed via the implantable medical lead, e.g., via one or more electrodes on the implantable medical lead. Even if the IMD is capable of storing physiological data, the storage space for physiological data may be limited. In addition, the physiological data recorder may include an electrode or sensor to sense physiological conditions of the patient that are not able to be sensed by the implantable lead or IMD.

Supplementing the electrical stimulation therapy functionality provided by an IMD with the lead extension may allow expansion or improvement of therapy efficacy and/or patient condition monitoring without replacement of the IMD or the medical lead. The lead extension with data recorder may be used to replace a previous lead extension. In other examples, a lead extension with data recorder according to this disclosure may be coupled to an existing lead and IMD where no lead extension had been previously used, or implanted with a new IMD.

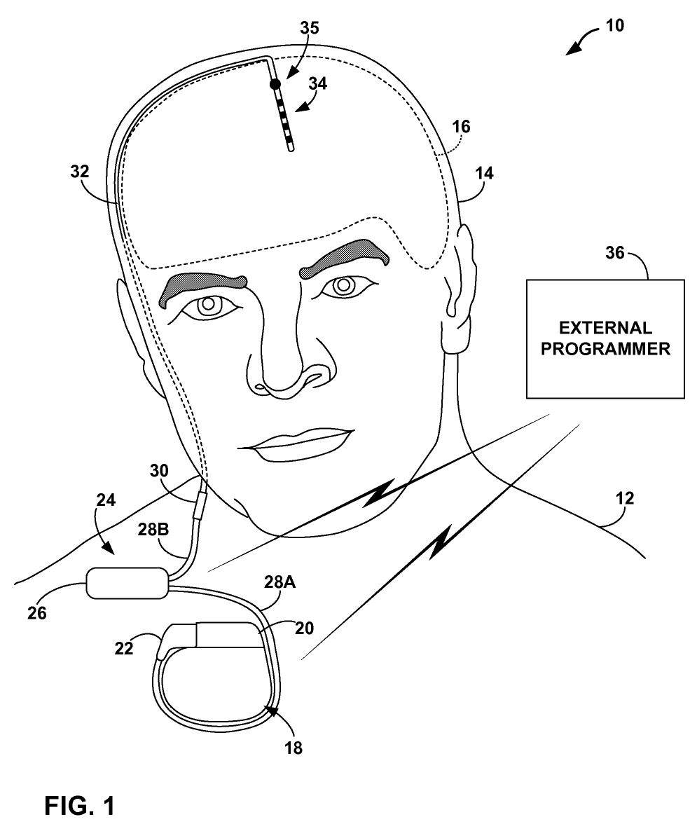

FIG. 1 is a conceptual diagram illustrating an example deep brain stimulation (DBS) system that includes an implantable lead extension 24 having a physiological data recorder 26. Although system 10 is a DBS system, systems that provide electrical stimulation therapy anywhere within patient 12 to treat any type of patient condition, e.g., chronic pain, gastric motility disorders, sexual dysfunction, incontinence, or obesity, may include lead extension 24.

As shown in FIG. 1, system 10 includes IMD 18, lead extension 24, medical lead 32, and external programmer 36. Lead extension 24 couples IMD 18 to lead 32. Lead extension 24 includes extension segments 28A and 28B. Extension segment 28A physically and electrically couples physiological data recorder 26 to IMD 18. Extension segment 28B physically and electrically couples physiological data recorder 26 to lead 32.

Extension segment 28A is electrically and physically coupled to IMD 18 via a connector 22 inserted within connector block 20 of IMD 18. Lead extension 24 traverses from the implant site of IMD 18 within a chest cavity of patient 12 to the proximal end of implanted medical lead 32. Extension segment 28B of lead extension 24 is electrically and physically coupled to lead 32 via a connector 30.

Lead 32 runs along the neck of patient 12 to cranium 14 of patient 12 to access brain 16. The distal end of lead 32 is implanted within brain 16 such that electrode array 34 is positioned adjacent to target areas for receipt of electrical stimulation therapy within brain 16. IMD 18 may deliver deep brain stimulation (DBS) therapy to patient 12 via one or more electrodes of array 34. Electrode array 34 may include ring electrodes, partial ring electrodes, or any other three-dimensional array of electrodes appropriate for treating patient 12.

In other examples, system 10 may include more than one lead 32 to place additional electrode arrays 34 within brain 16 or elsewhere within patient 12 for therapy or sensing purposes. If system 10 includes multiple leads attached to IMD 18, lead extension 24 may be configured to attach two or more leads 32 to IMD 18, or additional lead extensions 24 may couple the additional leads 32 to IMD 18. In some examples, lead extension 24 may be electrically and physically coupled to IMD 18 or lead 32 via an additional lead extension (not shown). In other words, as one example, IMD 18 may be coupled to another lead extension, and the other lead extension may be coupled to lead extension 24.

Physiological data recorder 26 receives a physiological signal sensed via one or more electrodes of array via lead 32 and extension segment 28B. Physiological data recorder 26 may process the signal to generate physiological data. The physiological data may include, for example, a brain signal. Examples of a brain signal include a recording of single cell action potentials, local field potentials (LFPs), an electrocorticogram (ECoG), or an electroencephalogram (EEG).

Physiological data recorder 26 may store the physiological data. Data recorder 26 may contain a large capacity to store data over a long period of time. For example, data recorder 26 may be capable of storing 128 megabytes (MB), 512 MB, 1 gigabyte (GB), 4 GB, 16 GB, or even more data as necessary for the monitoring of patient 12. Data may be stored on random access memory, flash memory, or any other type of electronic memory suitable to store the physiological data.

In some examples, lead 32 may include or be coupled to a sensor 35 in addition to electrode array 34. Physiological data recorder 26 may receive a physiological signal sensed by the sensor via lead 32 and extension segment 28B. Physiological data recorder 26 may process the signal to generate physiological data. Physiological data recorder 26 may store the physiological data.

In some examples, lead extension 24, e.g., data recorder 26, may include one or more additional electrodes (in addition to electrode array 34), one or more sensors, or both electrodes and one or more sensors. Physiological data recorder 26 may receive a physiological signal from the electrodes or sensors. Physiological data recorder 26 may process the signal to generate physiological data. Physiological data recorder 26 may store the physiological data. In some examples, physiological data recorder 26 may receive a physiological signal via a combination of one or more electrodes of array 34 and one or more electrodes on extension 24, e.g., on data recorder 26.

An example of a physiological signal received via additional electrodes on data recorder 26 is a far-field cardiac electrogram of patient 12. Sensors, such as sensor 35 on lead 32, or a sensor on lead extension 24 or within recorder 26, may include, as examples, a pressure sensor, such as a capacitive pressure sensor, a temperature sensor, an accelerometer, which may act as one or both of an activity sensor or a posture sensor, or any other sensor that detects a physiological condition of patient 12. Thus, the physiological data stored by data recorder 26 may include, as examples, blood or cerebral-spinal fluid (CSF) pressures, patient temperatures, patient activity data, or patient posture data.

Although data recorder 26 of lead extension 24 is shown implanted in the upper chest of patient 12, data recorder 26 may be implanted in any location desired by the clinician. Data recorder 26 may be implanted in a pocket created in an unobtrusive location within patient 12. Alternatively, data recorder 26 may be implanted such that an electrode or sensor of data recorder 26 is positioned to appropriately detect the desired patient condition.

Data recorder 26 is coupled to extension segments 28A and 28B on the same side of data recorder 26, but extension segments 28A and 28B may be located on different sides of the data recorder. For example, extension segments 28A and 28B may be located on opposite ends of data recorder 26 such that data recorder 26 lies in the path between IMD 18 and lead 32.

IMD 18 may deliver electrical stimulation via electrode array 34 to treat any number of neurological disorders or diseases. Example neurological disorders may include depression, dementia, obsessive-compulsive disorder, and movement disorders, such as Parkinson's disease, spasticity, and epilepsy. DBS is also useful for treating other patient conditions, such as migraines and obesity. In some examples, lead 32, and possibly IMD 18, may have been previously implanted and used to deliver DBS therapy to patient 12. Lead extension 24 may be introduced as a new component to record physiological data, either as a replacement to a previously used lead extension, or as an addition to the system 10. Lead extension 24 may be coupled to a newly implanted lead 32 or a previously implanted lead 32.

Lead 32 may be implanted within a desired location of brain 16 through a hole in cranium 14. Lead 32 may be placed at any location within brain 16 such that the electrodes of the leads are capable of providing electrical stimulation to targeted tissue during treatment. Example locations for electrode array 34 within brain 16 may include the pedunculopontine nucleus (PPN), thalamus, basal ganglia structures (e.g., globus pallidus, substantia nigra, subthalamic nucleus,), zona inserta, fiber tracts, lenticular fasciculus (and branches thereof), ansa lenticularis, and/or the Field of Forel (thalamic fasciculus).

In the case of migraines, as an example, lead 32 may be implanted to provide stimulation to the visual cortex of brain 16 in order to reduce or eliminate migraine headaches afflicting patient 12. In addition, as described in further detail below, electrode array 34 may be positioned to sense an EEG from within the visual cortex of brain. Such an EEG signal may be stored in data recorder 26.

In the case of obesity or compulsive-eating disorders, as another example, lead 32 may be placed to provide stimulation to provide negative feedback to patient 12, e.g., stimulating a sensory cortex of brain 16 to cause patient 12 to believe food tastes bad. However, the target therapy delivery site may depend upon the patient condition or disorder being treated.

IMD 18 includes a stimulation generator that generates the electrical stimulation delivered to patient 12 via lead 32. Generally the signal generator is configured to produce electrical pulses to treat patient 12. However, the signal generator of IMD 18 may additionally or alternatively be configured to generate a continuous wave signal, e.g., a sine wave or triangle wave.

In some examples, data recorder 26 may store physiological data from remote sensors that detect physiological conditions of patient 12. These remote sensors may communicate wirelessly with data recorder 26 to provide data recorder 26 with a physiological signal. Data recorder 26 may process the physiological signal and store physiological data, as described above.

Programmer 36 is an external computing device that the user, i.e., the clinician and/or patient 12, uses to communicate with IMD 18. Programmer 36 may be a hand-held computing device with a display viewable by the user and a user input mechanism that can be used to provide input to programmer 36. In other examples, programmer 36 may be a larger workstation or a separate application within another multi-function computing device. For example, the multi-function device may be a cellular phone or personal digital assistant that can be configured to execute an application providing medical device programmer functionality. Alternatively, a notebook computer, tablet computer, or other personal computer may execute an application to become a medical device programmer with a wireless communication adapter connected to the personal computer for communicating with IMD 18.

Programmer 36 may be used to transmit programming information to IMD 18, and to receive diagnostic or physiological information from IMD 18. The programming information may include therapy programs, which may specify values of one or more parameters of the therapy delivered by IMD 18. Example therapy parameters for electrical stimulation therapy include amplitude, duty cycle, and the combination or configuration of electrodes from array 34 through which IMD 18 delivers the stimulation. In the case of stimulation delivered as pulses, stimulation parameters may also include pulse width and rate. A clinician or patient may use programmer 36 to start, stop or modify the stimulation therapy delivered by IMD 18, e.g., to select stimulation therapy programs or modify the parameter values of a stimulation therapy program.

As shown in FIG. 1, programmer 36 may communicate with IMD 18 via wireless communication. Programmer 36, for example, may communicate via wireless communication with IMD 18 using radio frequency (RF) telemetry techniques known in the art. Programmer 36 may also communicate with another programmer or computing device via a wired or wireless connection using any of a variety of local wireless communication techniques, such as RF communication according to the 802.11 or Bluetooth specification sets, infrared communication according to the IRDA specification set, or other standard or proprietary telemetry protocols. Programmer 36 may also communicate with another programming or computing device via exchange of removable media, such as magnetic or optical disks, or memory cards or sticks. Further, programmer 36 may communicate with IMD 18 and other another programmer or computing device via remote telemetry techniques known in the art, communicating via a local area network (LAN), wide area network (WAN), public switched telephone network (PSTN), or cellular telephone network, for example.

As illustrated in FIG. 1, programmer 36 may also communicate wirelessly with data recorder 26 to retrieve any physiological data stored by the data recorder, or program the operation of data recorder 26 or a sensor of the data recorder. In other examples, programmer 36 may retrieve stored physiological data from data recorder 26 via a wired or other electrical contact connection when recorder 26 is explanted from patient 18. Furthermore, programmer 36 is but one example of an external device to which data recorder 26 may provide stored physiological data. In other examples, data recorder 26 may provide physiological data to any external computing device via a wireless or contact connection, which computing device need not provide medical device programmer functionality. In some examples, data recorder 26 may be provided with access to a network, e.g., via one or more external devices, and transmit stored physiological data to a remote server or database via the network.

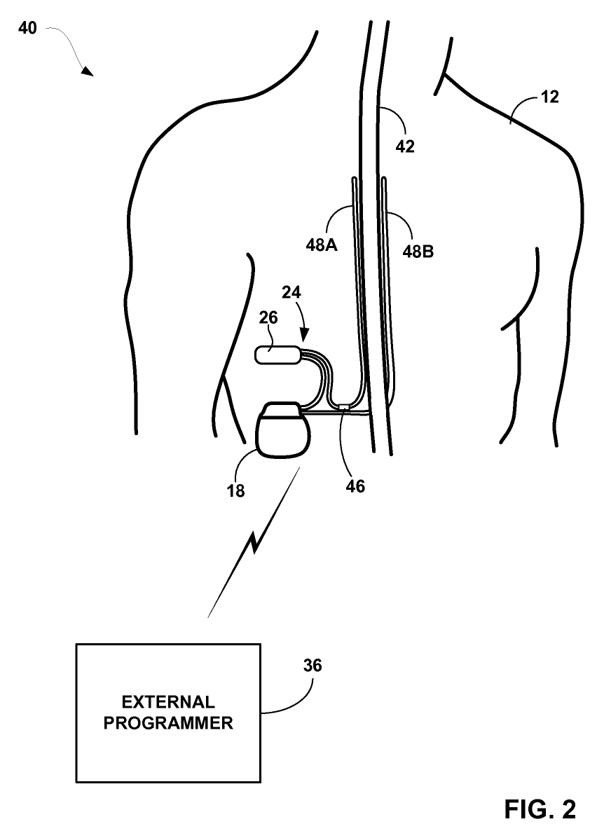

FIG. 2 is a conceptual diagram illustrating an example spinal cord stimulation (SCS) system 40 with lead extension 24 having a physiological data recorder 26. As shown in FIG. 2, IMD 18 is configured to deliver stimulation to spinal cord 42 via leads 48A and 48B (collectively “leads 48”) coupled to IMD 18. Lead 48A is coupled to IMD 18 via lead extension 24 having a physiological data recorder 26 configured to store data from patient 12. SCS system 40 is substantially similar to therapy system 10 of FIG. 1. However, rather than DBS, IMD 18 is configured to deliver SCS to spinal cord 42 of patient 12.

SCS therapy may be used, for example, to reduce pain experienced by patient 12. As another example, SCS therapy may be used to affect the performance of the heart or another organ of the patient. Furthermore, the lead extension 24 may be included in systems for delivery of other therapies instead of or in addition to DBS and SCS therapy, as illustrated in FIGS. 1 and 2. Other example therapies include delivery of stimulation to the pelvic nerves (not shown) or stomach (not shown) to treat incontinence, obesity, gastroparesis, or to any peripheral nerve, muscle or other tissue. In systems that provide any of these therapies, physiological data recorder 26 of lead extension 24 may store physiological data, which may indicate the progression or status of a condition of patient 12, the efficacy of the therapy, or the like.

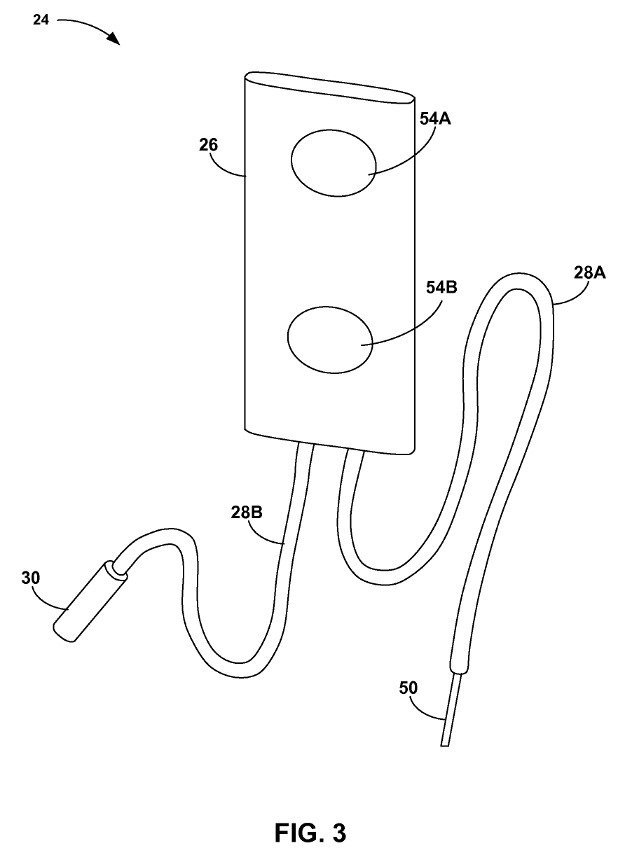

FIG. 3 is a conceptual diagram further illustrating an example configuration of lead extension 24 in which extension segments 28 are coupled to one side of physiological data recorder 26. As shown in FIG. 3, lead extension 24 includes extension segments 28A and 28B electrically and physically coupled to the same side of physiological data recorder 26. Extension segments 28A and 28B each include at least one flexible electrical conductor surrounded by an insulative and biocompatible flexible sheath, similar to leads and extensions common in the art. The one or more conductors within extension segments 28A and 28B may be straight or coiled within the protective sheath. In this manner, extension segments 28A and 28B may be capable of stretching with patient 12 movement to prevent conductor fracture.

Extension segment 28A includes pin 50 for insertion into a connector block 22 of IMD 18, i.e., for detachable coupling of extension segment 28A to IMD 18. Alternatively, pin 50 may fit into a connector of a secondary lead extension coupled to IMD 18 for detachable coupling to the other extension. In any case, pin 50 may be secured within connector block 22 or the secondary lead extension with set screws or any other fixation element. Pin 50 may include multiple contacts along the length of pin 50 to facilitate communication of a plurality of different electrical signals through a plurality of different conductions within extension segment 28A. Alternatively, extension segment 28A may include multiple pins.

Extension segment 28B includes connector 30 configured for accepting a pin from lead 32 or another lead extension, i.e., for detachable coupling of extension segment 28B to lead 32 or the other segment. Connector 30 may include a set strew, snap fit, threaded opening, or some other fixation element for securing the electrically coupled pin. Connector 30 may include multiple contacts along the length of connector 30 to facilitate communication of a plurality of different electrical signals through a plurality of different conductions within extension segment 28B.

Data recorder 26 is configured to store data representative of one or more physiological conditions of patient 12. The physiological data may be generated by a processor of data recorder 26 from sensed electrical signals. Data recorder 26 may be configured to generate physiological data from sensed electrical signals from electrode array 34 within brain 16 or any other therapy electrodes. Electrode array 34 may sense EEG signals, other brain signals, or any other signals that the clinician desires to store in data recorder 26 for later review. The processor within data recorder 26 may use the sensed electrical signals conducted through extension segment 28B to generate physiological data before storing the data. Data recorder 26 may also allow the sensed electrical signals to travel back to IMD 18 so as not to prevent normal IMD 18 function.

In addition, data recorder 26 may include one or more electrodes or sensors to detect a physiological condition of patient 12. In the example shown in FIG. 3, data recorder 26 includes electrodes 54A and 54B (collectively “electrodes 54”) on the housing of data recorder 26. Electrodes 54 on data recorder 26 may independently, or in conjunction with an electrode on IMD 18 or an electrode of electrode array 34, detect a cardiac electrogram of patient 12.

Data recorder 26 may store the sampled cardiac electrogram, or other data derived therefore, such as data regarding heart rate or morphological features of the cardiac electrogram. The clinician may, for example, later use the cardiac electrogram information and any events of brain 16 indicated by the EEG detected via electrode array 34 to determine how to modify DBS stimulation therapy. This functionality may not be possible without the inclusion of lead extension 24 in some systems including IMD 18.

In other examples, as discussed above, data recorder 26 may include or be coupled to a different type of sensor. Example sensors may include a pressure sensor, a temperature sensor, an ultrasound sensor, a flow sensor, or an activity or posture sensor. Such sensors may be located in or integrated with the housing of data recorder 26, tethered to data recorder 26, or in wireless communication with data recorder 26.

An example activity and/or posture sensor may include one or more accelerometers. In examples in which data recorder 26 includes such a sensor, data recorder may store data indicating patient exertion, movement, or posture over time. In such examples, a clinician may retrieve such information, as well as an EEG from electrode array 34, from data recorder 26. With such data, the clinician may, for example, be able to correlate any movement, exertion, or posture of patient 12 to EEG events or the like to analyze the progression of a patient condition or efficacy of DBS therapy. The clinician may identify modifications to the therapy based on such data.

Data recorder 26 may transmit any stored physiological data to external programmer 36 or another external computing device. The computing device may wirelessly request the physiological data when prompted by a user, such as a clinician. Data recorder 26 may then wirelessly transmit a portion or all of the stored physiological data to the computing device for the user to view.

Alternatively, data recorder 26 may be explanted for retrieval of the stored physiological data. In such examples, the housing of data recorder 26 may include electrical contacts, or a male or female connecter, to allow an electrical connection with an external computing device.

The housing of data recorder 26 may be configured to any desirable shape that fits within patient 12. Although data recorder 26 is shown as rectangular, data recorder 26 may be configured as cylindrical, ovular, spherical, cubical, or any other geometric shape. Data recorder 26 may be generally between 0.5 centimeters (cm) and 10 cm in length, 0.5 cm and 5 cm in width, and 0.2 cm and 3 cm in depth. More specifically, data recorder 26 may be between 2 cm and 5 cm in length, 1 cm and 3 cm in width, and 0.5 cm and 1.5 cm in depth. In addition, components of data recorder 26 may be sealed in a thin and flexible housing designed to be implanted just beneath the skin. For example, a flexible circuit with processor, memory, telemetry, and a power source may be spread out and coated with a flexible polymer to minimize any bulk beneath the skin of patient 12.

Extension segments 28A and 28B may be configured to any length and thickness necessary to implant lead extension 24 within patient 12. Although extension segments 28A and 28B may be substantially the same length, they may be different. Extension segments 28A and 28B may each be generally between 2 cm and 30 cm in length. More specifically, extension segments 28A and 28B may be between 10 cm and 20 cm in length. The thickness of extension segments 28A and 28B may be generally between 1 mm and 20 mm.

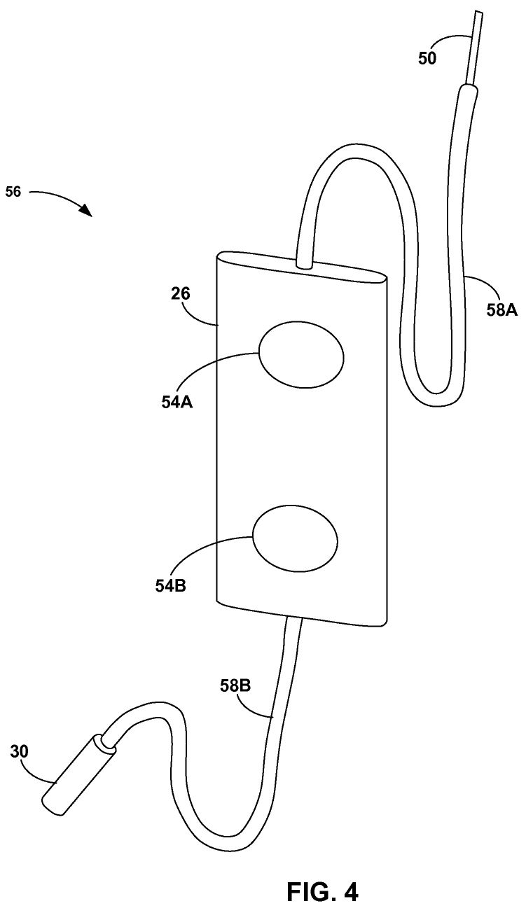

FIG. 4 is a conceptual diagram illustrating an example lead extension 56 having two extension segments 58A and 58B coupled to opposite sides of physiological data recorder 26. Lead extension 56 is substantially similar to lead extension 24 of FIG. 3. However, lead extension 56 is configured such that data recorder 26 is inline between extension segments 58A and 58B on opposite ends of data recorder 26. As illustrated in FIG. 4, extension segments 58A and 58B may be oriented along a longitudinal axis of data recorder 26. The configuration of extension segments 58A and 58B may be beneficial to implantation when the clinician desires to tunnel lead extension 56 beneath tissue of patient 12, because the longitudinal axes of segments 58 and recorder 26 are substantially coaxial.

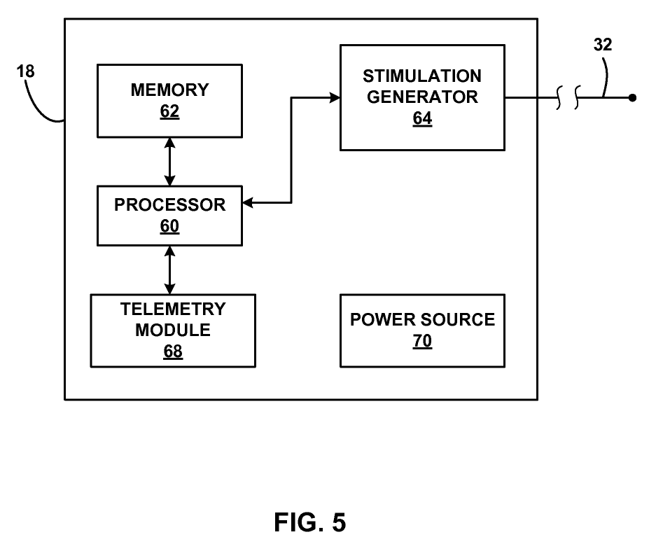

FIG. 5 is a functional block diagram illustrating components of IMD 18, according to one example. In the example of FIG. 5, IMD 18 includes processor 60, memory 62, stimulation generator 64, telemetry module 68, and power source 70. Memory 62 may include any volatile or non-volatile media, such as a random access memory (RAM), read only memory (ROM), non-volatile RAM (NVRAM), electrically erasable programmable ROM (EEPROM), flash memory, and the like. Memory 62 may store instructions for execution by processor 60, such as, but not limited to, therapy programs that control the delivery of stimulation therapy by IMD 18. In some examples, memory 62 stores program instructions that, when executed by processor 60, cause IMD 18 and processor 60 to perform the functions attributed to them herein.

Stimulation generator 64 is coupled to lead 32 via extension 24 (not shown in FIG. 5). Processor 60 controls stimulation generator 64 to deliver electrical stimulation therapy via lead 32 to electrode array 34 (not shown in FIG. 5). Processor 60 controls stimulation generator 64 according to therapy programs stored in memory 62. Processor 60 may include a microprocessor, a controller, a digital signal processor (DSP), an application specific integrated circuit (ASIC), a field-programmable gate array (FPGA), discrete logic circuitry, or the like.

Telemetry module 68 may accomplish communication by RF communication techniques. In addition, telemetry module 68 may communicate with programmer 36 via proximal inductive interaction with external programmer 36. Processor 60 controls telemetry module 68 to send and receive information.

Power source 70 delivers operating power to various components of IMD 18. Power source 70 may include a small rechargeable or non-rechargeable battery. Recharging may be accomplished through proximal inductive interaction between an external charger and an inductive charging coil within IMD 18.

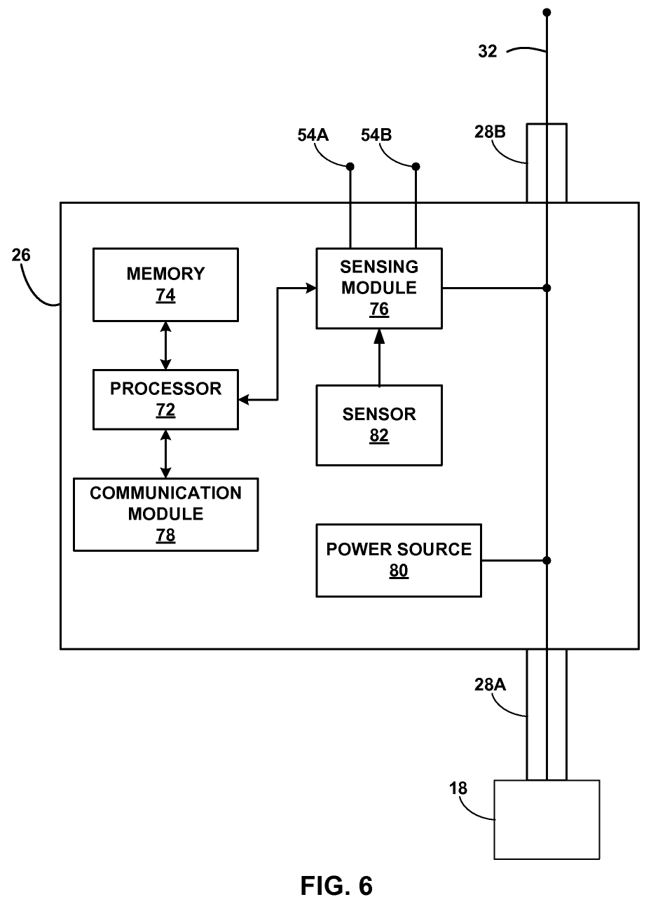

FIG. 6 is a functional block diagram illustrating components of physiological data recorder 26 according to one example configuration. In the illustrated example, data recorder 26 includes a processor 72, memory 74, sensing module 76, communication module 78, and power source 80. IMD 18, and more particularly stimulation generator 64 (FIG. 5), is coupled to lead 32 via conductors within extension segments 28 and data recorder 26. IMD 18 delivers electrical stimulation to patient via these conductors and lead 32. As illustrated in FIG. 6, sensing module 76 is coupled to lead 32, and power source 80 is coupled to IMD 18, via such conductors.

Sensing module 76 receives an electrical signal sensed by electrode array 34 (FIG. 1) via lead 32 and extension segment 28B. Processor 72 generates physiological data based on electrical signal received by the sensing module 76. Sensing module may include, as examples, amplifiers, filters, or other circuitry to condition the signal for processor 72.

The physiological data generated by processor 72 may include a digitized version of the signal. In such cases, processor 72 may comprise an analog-to-digital converter. The physiological data may additionally or alternatively include other information derived from the signal, such as minimum, maximum, range, mean, median, rate, or frequency values, or values associated with some morphological feature of the signal, as examples. As indicated above, the sensed signal and physiological data may be in the form of an EEG.

Sensing module 76 may also receive an electrical signal sensed via electrodes 54, or a physiological signal from a sensor 82. An example of a signal that may be sensed via electrodes 54 is a cardiac electrogram signal. An example of a sensor 82 is an accelerometer, which may generate one or more signals that vary as a function of patient activity or posture. Other examples of physiological signals and sensors have been described above. Sensing module 76 may condition such signals for use by processor 72, and processor 72 may generate physiological data based on such signals, in the manner described above with respect to the signal received from lead 32.

Processor 72 stores generated physiological data within memory 74. Memory 74 may include any volatile or non-volatile media, such as a random access memory (RAM), read only memory (ROM), non-volatile RAM (NVRAM), electrically erasable programmable ROM (EEPROM), flash memory, or the like. Memory 74 may be configured to store large amounts of physiological data that covers days, weeks, months, or years of patient 12 physiological data. Storage capacities of memory 74 may be generally between 128 KB and 250 GB. Of course, larger capacities are commonly being developed, and any size that satisfies the implantable nature of data recorder 26A may be appropriate. Preferably, solid state memory, such as flash memory, would be employed as memory 74 to limit power consumption, size, and the probability of mechanical failure.

Communication module 78 may accomplish communication with programmer 36 or another external computing device by wireless, e.g., RF or proximal inductive, communication. In such examples, communication module may include or be coupled to an antenna or coil. In other examples, as discussed above, the communication module 78 may be configured for communication with device electrically coupled to the communication module. Processor 72 controls the communication by communication module 78.

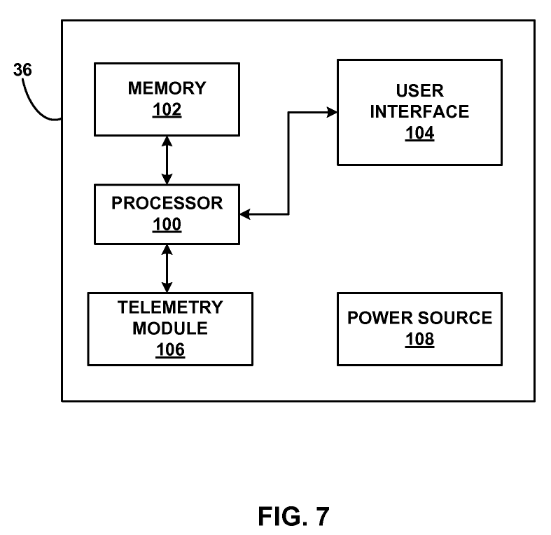

Power source 80 may be primary or rechargeable, and may include, for example, a battery or a capacitor. In some examples, power source 80 may be charged by proximal inductive interaction with an external charger. In the example illustrated by FIG. 7, power source 80 is electrically coupled to IMD 18 via conductors within data recorder 26 and extension segment 28A. In such examples, power source 80 may be charged by IMD 18, and more particularly, by signals generated on the conductors by stimulation generator 64 of IMD 18.

The signals provided by IMD 18 may be signals not intended for therapy, e.g., the parameters of these signals may be different than those intended to provide electrical stimulation to patient 12. IMD 18 may be programmed to periodically emit signals that power source 80 can store. In other examples, power source 80 may be charged by therapeutic stimulation signals delivered by IMD 18. Power source 80 may include rectification circuitry that rectifies the signals from IMD 18 for charging the power source. For example, therapy signals, such as biphasic pulses, may be half-wave rectified, and the rectified signal used to charge power source 80.

FIG. 7 is functional block diagram illustrating components of external programmer 36 according to one example configuration. In the illustrated example, external programmer 36 includes a processor 100, memory 102, user interface 104, telemetry module 106, and power source 108. Processor 100 controls user interface 104 and telemetry module 106, and stores and retrieves information and instructions to and from memory 102. Programmer 36 may be configured for use as a clinician programmer or a patient programmer.

Programmer 36 may be used to select therapy programs (e.g., sets of stimulation parameters), generate new therapy programs, modify therapy programs through individual or global adjustments, transmit the new programs to a medical device, such as IMD 18, and communicate with data recorder 26 of lead extension 24 or 56. Programmer 36 may define sensing and storing instructions for data recorder 26 and retrieve any stored physiological data. Alternatively, programmer 36 may communicate with and retrieve data from data recorder 26 via IMD 18.

The user, either a clinician or patient 12, may interact with programmer 36 through user interface 104. User interface 104 includes a display (not shown), such as an LCD or other screen, to show information related to stimulation therapy and input controls (not shown) to provide input to programmer 36. Processor 100 monitors activity from the input controls and controls the display or stimulation function accordingly. In some embodiments, the display may be a touch screen that enables the user to select options directly from the display. In other embodiments, user interface 104 also includes audio circuitry for providing audible instructions or sounds to patient 12 and/or receiving voice commands from patient 12.

Memory 102 may include instructions for operating user interface 104, telemetry module 106 and managing power source 108. Memory 102 also includes instructions for managing physiological data from data recorder 26, such as further processing of the physiological data, creation of graphs or tables, or any other data manipulation available to the user. The clinician may use this physiological data to determine the progression of patient 12 disease in order to predict future treatment.

Memory 102 may include any volatile or nonvolatile memory, such as RAM, ROM, EEPROM or flash memory. Memory 102 may also include a removable memory portion that may be used to provide memory updates or increases in memory capacities. A removable memory may also allow sensitive patient data to be removed before programmer 36 is used by a different patient. Processor 100 may comprise any combination of one or more processors including one or more microprocessors, DSPs, ASICs, FPGAs, or other equivalent integrated or discrete logic circuitry. Accordingly, processor 100 may include any suitable structure, whether in hardware, software, firmware, or any combination thereof, to perform the functions ascribed herein to processor 100.

Wireless telemetry in programmer 36 may be accomplished by RF communication or proximal inductive interaction of external programmer 36 with IMD 18 or data recorder 26. This wireless communication is possible through the use of telemetry module 106. Accordingly, telemetry module 106 may be similar to the telemetry module contained within IMD 18 or data recorder 26. In alternative embodiments, programmer 36 may be capable of infrared communication or direct communication through a wired connection. In this manner, other external devices may be capable of communicating with programmer 36 without needing to establish a secure wireless connection.

Power source 108 delivers operating power to the components of programmer 36. Power source 108 may include a battery and a power generation circuit to produce the operating power. In some embodiments, the battery may be rechargeable to allow extended operation. Recharging may be accomplished electrically coupling power source 108 to a cradle or plug that is connected to an alternating current (AC) outlet. In addition, recharging may be accomplished through proximal inductive interaction between an external charger and an inductive charging coil within programmer 36. In other embodiments, traditional batteries (e.g., nickel cadmium or lithium ion batteries) may be used. In addition, programmer 36 may be directly coupled to an alternating current outlet to operate. Power source 108 may include circuitry to monitor power remaining within a battery. In this manner, user interface 104 may provide a current battery level indicator or low battery level indicator when the battery needs to be replaced or recharged. In some cases, power source 108 may be capable of estimating the remaining time of operation using the current battery.

In other embodiments, functions of programmer 36 may be employed remotely. IMD 18 and/or data recorder 26 may interact with a communication device that transmits data over a network. The network may include a server that distributes the data to any remote device used by a clinician, technician, or another user. In this manner, patient 12 condition may be able to be remotely monitored to reduce the frequency of patient 12 visits to a clinic and improve the treatment of patient 12.

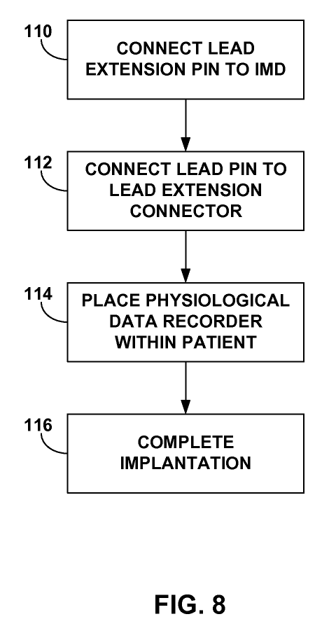

FIG. 8 is a flow diagram illustrating an example technique for connecting a lead extension 24 having a physiological data recorder 26 to a lead and IMD 18. Although the method of FIG. 8 is described with regard to lead extension 24, lead extension 56 or any other lead extension may be used. In addition, lead extension 24 is described as being implanted and coupled to a previously implanted lead 32 and IMD 18, lead extension 24 may similarly be implanted with a new lead 32 and IMD 18 at the time of their implant.

A clinician may perform the implantation procedure of FIG. 8. The clinician connects pin 50 of lead extension 24 to IMD 18 (110). The clinician connects the pin of lead 32 to connector 30 of lead extension 24 (112). At this time, the clinician may ensure that lead extension 24 is operational with IMD 18 and lead 32.

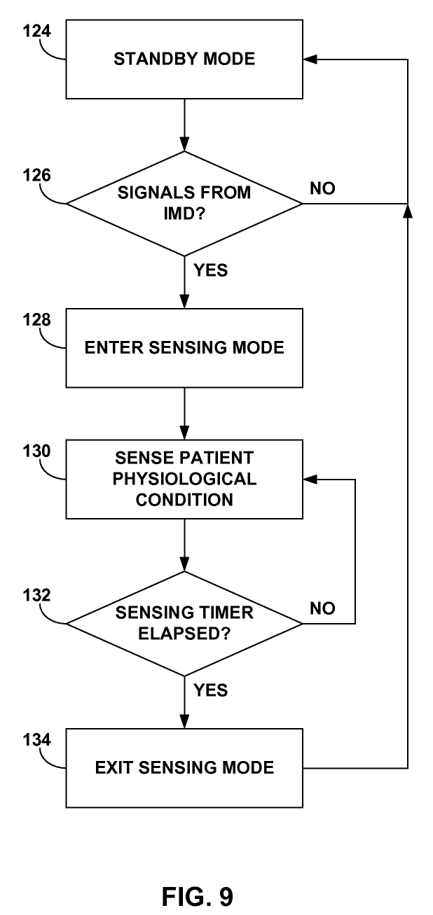

FIG. 9 is a flow diagram illustrating an example technique for a physiological data recorder 26 to enter a sensing mode in response to detecting stimulation therapy signals from IMD 18. Although data recorder 26 may store physiological data periodically or continuously at any time when implanted in patient 12, a clinician may only desire to monitor patient 12 conditions during therapy delivery. As shown in FIG. 9, processor 72 of data recorder 26 is normally operating in a standby mode (124). Standby mode may require minimal power and only minimal processing by processor 72. Some periodic generation of physiological data may be performed during the standby mode. Until processor 72 detects signals from IMD 18 (126), processor 72 continues to operate in standby mode (124).

Upon detecting stimulation signals from IMD 18 (126), or electrical signals having parameters similar to those used for therapy, via sensing module 76 and the conductors within extension 24, processor 72 exits the standby mode and enters the sensing mode (128). Processor 72 then operates data recorder 26 to generate and store physiological data representative of a physiological condition of patient 12 (130). Unless the sensing timer elapses (132), processor 72 continues to generate physiological data (130). Each time that processor 72 detects stimulation signals from IMD 18, processor 72 re-starts the sensing timer. The sensing timer is set to a predefined period such that processor 72 re-enters the standby mode after stimulation therapy ceases. Generally, the sensing timer may have a duration between 60 seconds and 24 hours. Once the sensing timer elapses (132), processor 72 exits the sensing mode and re-enters the standby mode (124) until subsequent stimulation signals are detected.

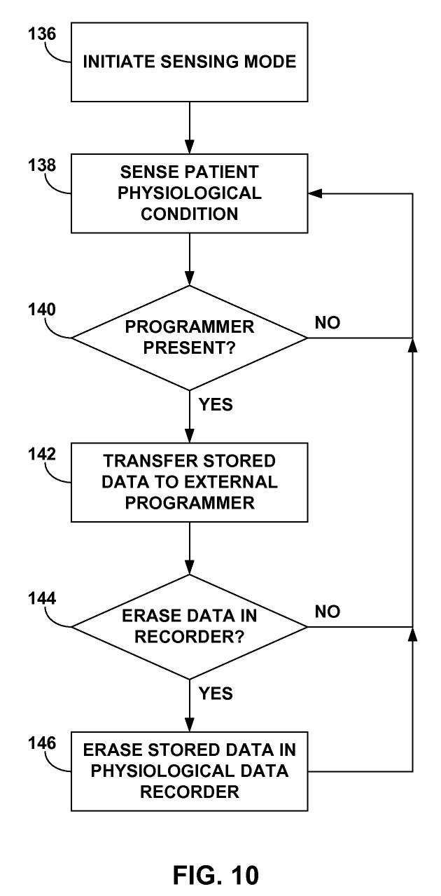

FIG. 10 is a flow diagram illustrating an example technique for wirelessly transferring physiological data from physiological data recorder 26 to external programmer 36. Although described with respect to external programmer 36, the method of FIG. 10 may be performed to transfer physiological data from data recorder 26 to any external computing device.

As shown in FIG. 10, data recorder 26 enters a sensing mode (136). Once in sensing mode, processor 72 of data recorder 26 generates physiological data from sensed electrical signals and stores the generated data in memory 74 (138). Unless processor 72 determines that programmer 36 is present and receives a request to change operation (140), processor 72 continues to store physiological data (138).

If processor determines that programmer 36 is present (140), processor communicates with programmer 36 and transfers part or all of the stored physiological data to programmer 36 (142). In some examples, processor 72 may only wirelessly transfer certain requested physiological data, i.e., data for a certain time period. Once the physiological data is transferred, processor 72 determines if that data just transferred should be erased from memory 74 (144). If the data is to be erased (144), processor 72 erases the transferred data from memory 74 (146) and continues to operate in sensing mode to store physiological data (138). Processor 72 may have instructions to erase any transferred physiological data in order to create storage space for newly generated data. Alternatively, processor 72 may only erase transferred physiological data when less than a predetermined amount of memory space remains.

Many examples according to the disclosure have been described. Various modifications may be made to these examples without departing from the scope of the claims. These and other examples are within the scope of the following claims.

1. An implantable lead extension comprising:
a first extension segment;
a second extension segment; and
a physiological data recorder electrically and physically coupled to the first and second extension segments,
wherein the first extension segment is configured to electrically and physically couple the physiological data recorder to one of an implantable medical device (IMD) or another lead extension,
wherein the second extension segment is configured to electrically and physically couple the physiological data recorder to one of an implantable medical lead having at least one lead electrode or the other lead extension, and
wherein the physiological data recorder comprises:
a memory,
a communication module, and
a processor configured to receive a physiological signal of a patient via the at least one electrode of the implantable medical lead, generate physiological data based on the physiological signal, store the physiological data within the memory, and provide the stored physiological data to an external device via the communication module.

2. The implantable lead extension of claim 1, wherein the physiological data comprises a brain signal.

3. The implantable lead extension of claim 1,
wherein the physiological signal comprises a first physiological signal and the physiological data comprises first physiological data,
wherein the implantable medical lead further comprises at least one sensor, and
wherein the processor is configured to receive a second physiological signal of the patient from the at least one sensor of the implantable medical lead, generate second physiological data based on the second physiological signal, store the second physiological data within the memory, and provide the stored second physiological data to the external device via the communication module.

4. The implantable lead extension of claim 1,
wherein the physiological signal comprises a first physiological signal and the physiological data comprises first physiological data,
wherein the lead extension comprises at least one of another electrode or a sensor, and
wherein the processor is configured to receive a second physiological signal of the patient from the at least one of the other electrode or the sensor of the lead extension, generate second physiological data based on the second physiological signal, store the second physiological data within the memory, and provide the stored second physiological data to the external device via the communication module.

5. The implantable lead extension of claim 4, wherein the physiological data recorder comprises the at least one of the other electrode or the sensor.

6. The implantable lead extension of claim 4, wherein the sensor comprises at least one of a pressure sensor, a temperature sensor, or an accelerometer.

7. The implantable lead extension of claim 4, wherein the second physiological data comprises at least one of a cardiac electrogram, a blood pressure, a cerebral-spinal fluid pressure, a patient temperature, patient activity data, or patient posture data.

8. The implantable lead extension of claim 1, wherein the physiological data recorder comprises a power source that is configured to be charged by electrical signals generated by a stimulation generator within the IMD and received via the first extension segment.

9. The implantable lead extension of claim 1, wherein communication module comprises telemetry circuitry and an antenna, the telemetry circuitry and antenna configured to transmit the stored physiological data to an external device via wireless telemetry when the physiological data recorder is implanted.

10. The implantable lead extension of claim 1, wherein the first extension segment is coupled to a first side of the physiological data recorder and the second extension segment is coupled to a second side of the physiological data recorder.

11. The implantable lead extension of claim 1, wherein the first extension segment and the second extension segment are coupled to a common side of the physiological data recorder.

12. A method comprising:
receiving a physiological signal via at least one electrode of an implanted medical lead with an implanted physiological data recorder of an implanted lead extension, wherein a first extension segment of the extension is coupled to the implanted medical lead and a second extension segment of the extension is coupled to an implanted medical device that delivers electrical stimulation therapy via the implanted lead;
generating physiological data with the physiological data recorder based on the physiological signal;
storing the physiological data in a memory of the physiological data recorder; and
providing the stored physiological data from the physiological data recorder to an external device.

13. The method of claim 12, wherein the physiological data comprises a brain signal.

14. The method of claim 12,
wherein the physiological signal comprises a first physiological signal and the physiological data comprises first physiological data, and
wherein the implanted medical lead further comprises a sensor,
the method further comprising:
receiving a second physiological signal of the patient from the sensor of the implanted medical lead with the physiological data recorder;
generating second physiological data with the physiological data recorder based on the second physiological signal;
storing the second physiological data within the memory of the physiological data recorder; and
providing the stored second physiological data from the physiological data recorder to the external device.

15. The method of claim 12,
wherein the physiological signal comprises a first physiological signal and the physiological data comprises first physiological data, and
wherein the physiological data recorder comprises at least one of another electrode or a sensor,
the method further comprising:
receiving, with the physiological data recorder, a second physiological signal of the patient from the other electrode or sensor of the physiological data recorder;
generating second physiological data with the physiological data recorder based on the second physiological signal;
storing the second physiological data within the memory the physiological data recorder; and
providing the stored second physiological data from the physiological data recorder to the external device.

16. The method of claim 15, wherein the sensor comprises at least one of a pressure sensor, a temperature sensor, or an accelerometer.

17. The method of claim 15, wherein the second physiological data comprises at least one of a cardiac electrogram, a blood pressure, a cerebral-spinal fluid pressure, a patient temperature, patient activity data, or patient posture data.

18. The method of claim 12, further comprising charging a power source of the physiological data recorder from electrical signals generated by a stimulation generator of the implanted medical device.

19. The method of claim 12, wherein providing the stored physiological data from the physiological data recorder to the external device comprising wirelessly transmitting the physiological data from the physiological data recorder to the external device.

20. A system comprising:
an implantable medical lead having at least one electrode;
an implantable medical device (IMD) configured to deliver electrical stimulation therapy to a patient via the at least one electrode of the lead; and
an implantable lead extension comprising:
a first extension segment,
a second extension segment, and
a physiological data recorder electrically and physically coupled to the first and second extension segments,
wherein the first extension segment is configured to electrically and physically couple the physiological data recorder to one of the IMD or another lead extension,
wherein the second extension segment is configured to electrically and physically couple the physiological data recorder to one of the implantable medical lead or the other lead extension, and
wherein the physiological data recorder comprises:
a memory,
a communication module, and
a processor configured to receive a physiological signal of a patient via the at least one electrode of the implantable medical lead, generate physiological data based on the physiological signal, store the physiological data within the memory, and provide the stored physiological data to an external device via the communication module.

21. The system of claim 20, wherein communication module comprises telemetry circuitry and an antenna, the telemetry circuitry and antenna configured to transmit the stored physiological data to an external device via wireless telemetry when the physiological data recorder is implanted.

22. The system of claim 20, wherein the physiological data recorder comprises a power source that is configured to be charged by the electrical stimulation generated by the IMD.
